(19) United States
(12) Patent Application Publication
(10) Pub. No.: US 2011/0258869 A1
(43) Pub. Date: Oct. 27, 2011

(54) DEVICE FOR DETERMINING MEASUREMENTS OF HUMAN BODY PARTS

(75) Inventor: Erwin Bittkowski, Wertheim (DE)

(73) Assignee: GANONI & CIE AG, ST GALLEN (CH)

(21) Appl. No.: 12/280,057

(22) PCT Filed: Feb. 13, 2007
(86) PCT No.: PCT/DE07/00300

§ 371 Date: Aug. 20, 2008

(30) Foreign Application Priority Data
Feb. 22, 2006 (DE) 20-2006-002-947.8

(51) Int. Cl. A61B 5/103; G01B 11/24

(52) U.S. Cl. 33/512; 356/625

(57) ABSTRACT

The invention relates to a device for determining measurements of human body parts (B), in particular the leg and hip measurements to be determined for the supply with compression stockings, at which first measuring means (5) are provided for measuring the circumference of said body part (B), whereat the device comprises second measuring means (6, 7), with which the length section (1C) of said body part (B) can be determined from a reference plane up to a circumferential plane (U).

The invention relates to a device for determining measurements of human body parts according to the preamble of claim 1. Such devices are in particular adapted for determining the leg and hip measurements to be determined for a supply with compression stockings.

Medical compression stockings, which are often used subsequent to operations to avoid thrombosis risks, are manufactured from elastic materials, and after donning them, they exert pressure on the sheathed tissue, causing vascular compression, which prevents, or at least inhibits, the formation of blood clots. An exact fit is indispensable for the optimal therapeutic effect of compression stockings. Therefore, the precise determination of the leg and hip measurements is of decisive significance for the fit of compression stockings. When taking the measurements for the supply with compression stockings, it has been common so far to first measure the circumferences of the legs and the hip at standardized measuring points with a measuring tape, and then, using the measuring tape, to determine the individual measures of length associated with the circumferences. In such a measuring process, errors in the length measurement easily occur. It is furthermore disadvantageous that the measuring process is relatively time-consuming and complicated.

The object of the present invention now consists in suggesting a device for determining measurements of human body parts, in which the disadvantages stated above are eliminated at least to a large extent.

This object is solved with the features of claim 1.

The device for determining measurements of human body parts comprises first measuring means for measuring the circumference of the body part. According to the invention, the device comprises second measuring means, with which the length section of the body part can be determined from a reference plane up to the circumferential plane.

Preferably, with the second measuring means the distance measured between the circumferential plane and the reference plane in the direction of the longitudinal extension of the body part, generally vertical to the circumferential plane, can be measured.

Preferably, with the second measuring means the distance can be measured in a contactless manner, whereat for the contactless distance measurement basically any known optical and acoustical methods may be applied.

In a particular embodiment, the device comprises a sender for emitting a physical measurement signal. The reference basis is designed as a reflection plane generally arranged in parallel to the circumferential plane, at which reflection plane the signal can be reflected. Furthermore, the device comprises a receiver for receiving the measurement signal reflected at the reflection surface as well as an evaluation unit for determining the distance on the basis of the time delay of the measurement signal.

Preferably, the measurement signal is an acoustic signal, in particular an ultrasonic signal.

Since, as is generally known, the speed of sound depends on the temperature of the ambient air, according to a further advantageous embodiment, the device comprises means for compensating temperature-related variations in the speed of sound, which comprise a temperature sensor and an evaluation unit.

In a particular embodiment, the measurement signal is an optical signal, in particular an optical signal generated by a laser. The optical signal is emitted by the sender, reflected at the reflection surface, and reflected back to the receiver. From the time delay of the signal, the distance between the circumferential plane and the reference plane is determined in a known manner using an electronic evaluation unit.

The device preferably comprises a display for displaying the measured values. Basically, any known indications or displays are suitable for that, like in particular LED (light-emitting diode) displays.

In particular, the device comprises an interface for data transfer, via which the measurement data comprising the circumferences and the respectively associated length sections can be transferred to a computer for further processing. For example, the interface is a USB (universal serial bus) system for connecting the device with external peripheral devices for the exchange of data.

Preferably, the interface is designed bidirectional, i.e. via the interface data can be transferred from a signal processing unit in the device to an external computer as well as in the opposite direction from an external computer to the signal processing unit. Thus it is possible to allocate the measured body measurements to individual, personal data of the patient.

In a particularly advantageous embodiment of the device, the first measuring means comprises a measuring tape loop, which is to be positioned around the circumference to be measured of the body part to be measured.

Preferably, the circumference of the measuring tape loop can be varied.

For example, the circumference of the measuring tape loop can be varied using an electromotive drive.

In one embodiment, the measuring tape loop is connected with a wind-up unit comprising a spring, which is tensioned upon the measuring tape loop being pulled out. The measuring tape is automatically wound up onto a coil of the wind-up unit by the force of the spring connected with one end of the measuring tape.

Preferably, means are provided to stop the pull-in movement of the measuring tape loop upon reaching a predetermined loop contact pressure.

Preferably, the measuring tape loop is provided with a fastening means, by which two loop sections can be releasably connected. As the fasting means, basically any fastening means are possible, such as Velcro fasteners, button fasteners, or the like.

In one embodiment, the measuring tape loop is provided with an incremental gauge, whereat additionally a scanning unit is provided, with which the circumference of the measuring tape loop can be measured by scanning the gauge. Basically, the use of any known magnetic, optical, or inductive incremental measuring systems is conceivable.

Preferably, the gauge is designed as punching or toothing.

Preferably, the scanning unit is an optical scanning unit, which comprises at least one light emitter and one photo-detector.

Preferably, the device is provided with computing means, with which the counting pulses generated by the scanning unit upon a pull-in movement of the measuring tape loop and the counting pulses generated by the scanning unit upon an extension movement of the measuring tape loop can be added up, and with which, on the basis of the result of the addition, the current circumference of the measuring tape loop can be determined. In this case, no absolute measure is provided on the measuring tape.

Preferably, the computing means comprise a differential encoder in connection with the photo-detector.

In a particular embodiment, calibration means are provided for zero-value calibration of the measuring tape loop. The calibration preferably takes place software-supported on the basis of a reference circumference.

Preferably, the device comprises a housing, in which, among others, the computing and/or evaluation units for the evaluation and, if applicable, output of the measurement signals are accommodated.

Preferably, at least one inclination sensor for determining the relative position between the housing and the reference plane is provided at the housing.

Possible inclination sensors include liquid-based inclination sensors, with which the horizon of a liquid is optically, resistively, magnetically, or capacitively scanned, as well as micromechanical inclination sensors, with which the deflection of a mass suspended from a spring or a spring bar is scanned. Naturally it is also conceivable to provide one or several spirit levels.

Preferably, the side of the housing facing the body part comprises an inwardly concave outer contour complementary to the body part, which enables the at least almost free-of-play fit of the housing at the body part.

Preferably, the device is provided with an instruction unit, with which instructions for the execution of the measuring process can be transmitted to a user. The instruction unit may, for example, comprise a software program, with which instructions for the execution of the measurement are displayed to the user via a screen.

In particular, the instruction unit is provided with an acoustic announcement, which is connected with a sound storage device and transmits instructions about the execution of the measurement to the user in the form of spoken text.

Preferably, the device is provided with a foot length measuring unit.

In particular, the housing is designed as a foot length measuring unit.

In one embodiment, the foot length measuring unit additionally comprises an extension part, which can be slid out of the housing or folded out from the housing.

In a further embodiment, the device comprises an input unit, in particular in the form of a keyboard, via which a signal processing unit and/or a data storage in the device can be supplied with information. Via this input unit, e.g. personal data of patients can be entered directly before the measurement, so that, in particular if several patients are to be measured in succession, an allocation of patient data to the respective measurement results is possible.

Preferably, the device is designed to be able to execute the operations required for the processing of measurement signals and/or of data entered via the input unit as well as for the output and/or display of the measurement results autarkically and independently from external computers or peripheral devices. I.e., the device is preferably designed as a stand-alone device, and for its routine application as intended does not require any external EDP devices like PCs or screens. For example, it is conceivable that an evaluation unit in the device can decide on the basis of characteristics of the stockings supplier, whether for a measured patient a standard stocking can be used, and, if applicable, issue the identity designation of the standard stocking.

The invention has the advantage that compared to the state of the art it allows for a substantially faster and more precise determination of the measurements of human body parts.

In the following, a preferred embodiment of the device is explained in more detail on the basis of the drawings, in which:

FIG. 1 is a device according to the invention in a schematic representation in perspective;

FIG. 2 shows the positions of anatomically standardized measuring points in the area of the lower extremities and the hip in a schematic representation;

FIG. 3 shows the use of the device according to the invention as exemplified by a calf measurement in a schematic representation; and

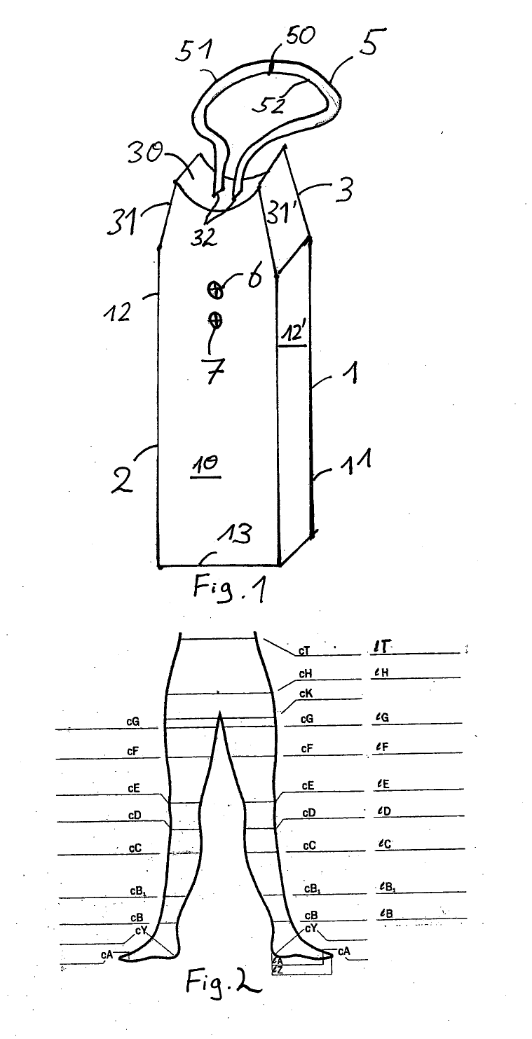

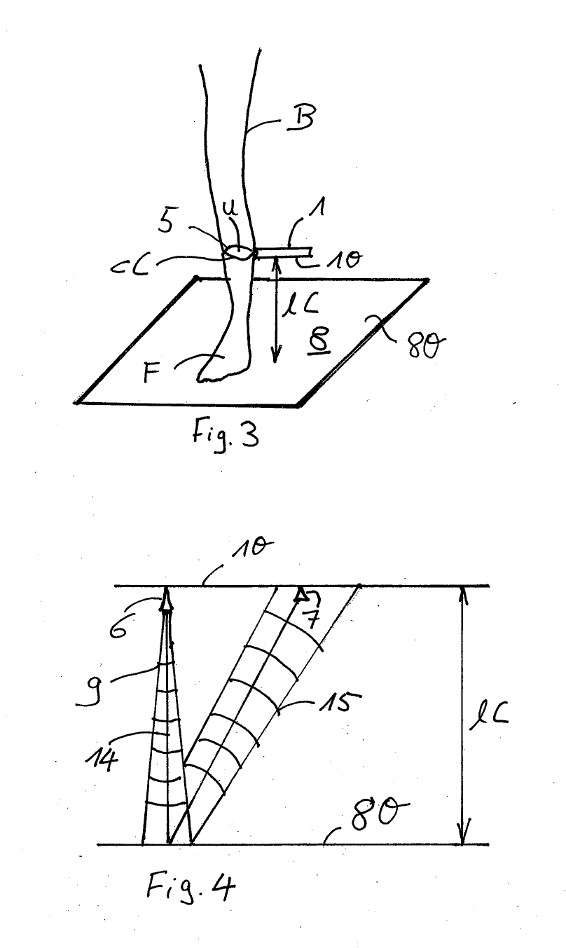

FIG. 4 shows the distance determination executed for the measurement according to FIG. 3 in a schematic diagram.

FIG. 1 shows a device according to the invention in a schematic representation in perspective, which comprises a housing 1, in which, among others, electronic computing and evaluation units (not shown) for the evaluation and, if applicable, the output of measurement signals received are accommodated. Furthermore, electronic data storage means (not shown) are located within the housing 1.

Firstly, the housing 1 consists of a front side 10 and a rear side 11 arranged in parallel and at a distance to the latter, which are laterally connected by two side faces 12, 12′. The base 13 defines the housing 1 on its side facing away from the body part.

The housing 1 comprises a cuboid-shaped section 2, followed by a head section 3, the contact surface 30 of which facing the body part is inwardly concave, and which head section is laterally defined by two flank faces 31, 31′ converging towards the contact surface 30.

The contact surface 30 is provided with two slots 32 arranged at a distance, through which the loop-shaped wound measuring tape loop 5 passes. Within the housing 1, an electromotive drive (not shown) is provided, which comprises a winding shaft connected with one end of the measuring tape loop 5, onto which the measuring tape loop 5 can be wound up and can be drawn into the housing varying its circumference.

The measuring tape loop 5 comprises two loop sections 51, 52, the free ends of which are releasably connected by a fastening means 50. With the fastening means 50, which, e.g., is a Velcro fastener, the measuring tape loop 5 can be opened and closed.

The maximum pressure to be exerted onto the body part upon drawing in the measuring tape loop 5 is limited by an electronic power control of the electromotive drive.

Naturally, the limitation of the maximum contact pressure may also take place with other means, which fulfill the same purpose, such as, e.g., by a friction clutch (not shown) arranged between the wind-up shaft and the drive, which effects that upon reaching a predetermined maximum loop contact pressure the draw-in movement of the measuring tape loop 5 is stopped.

The measuring tape loop 5 is provided with an incremental gauge (not shown) in the form of a punching with holes arranged at uniform distances. Furthermore, an optical scanning unit (not shown) is provided within the housing 1, which comprises a light emitter arranged on one side of the measuring tape, e.g. in the form of an LED light source, and a photo-detector arranged on the other side of the measuring tape, which detects the light entering through respectively one hole of the punching.

At the front side 10, an ultrasonic sender/receiver system 6, 7 is arranged, the sender 6 of which comprises a piezoelectric oscillator (not shown), which generates a sound signal in the frequency range of about 40 kHz, which is inaudible for the human being. Sound frequencies in this range can be bundled relatively well and propagate in a cone shape.

FIG. 2 shows a schematic representation with the positions of the anatomically standardized measuring points in the area of the lower extremities and the hip, as they are known to the skilled person. The likewise standardized abbreviations in detail stand for:

1. Circumferential Measurements:
cA: Circumference of the foot at the metatarsophalangeal joint
cY: Circumference over heel and instep (foot bent)
cB: Circumference of the ankle
cB1: Circumference of the base of the calf
cC: Circumference of the calf
cD: Circumference below the knee
cE: Circumference of the knee joint
cF: Circumference of the base of the thigh muscles
cG: Circumference of the top of the thigh
cH: Circumference of the hip
cT: Circumference of the waist

2. Length Measurements:
1A: Length section from heel to base of the toes
1B: Length section from heel to ankle
1B1: Length section from heel to base of the calf
1C: Length section from heel to calf
1D: Length section from heel to below the patella
1E: Length section from heel to knee
1F: Length section from heel to start of the thigh
1G: Length section from heel to end of the thigh
1H: Length section from heel to hip
1T: Length section from heel to waist

FIG. 3 shows the use of the device according to the invention exemplified by the determination of the circumference of the calf cC as well as the associated length section 1C in a schematic representation. The foot F of a patient is positioned on a horizontally arranged base-plate 8 having a plane, upward facing upper side 80, which serves as the reference plane for the measurement of the length section 1C. The leg B should be as extended as possible and vertically oriented to the base-plate 8, i.e. the geometric axis defined by the longitudinal extension of the leg B should generally be vertically oriented to the plane defined by the base-plate 8. The front side 10 of the housing 1 is facing the base-plate 8, and with the aid of inclination sensors (not shown) connected with the housing 1, it is oriented in parallel to the base-plate 8. The circumferential plane U at the measuring point cC is generally oriented in parallel to the front side 10 as well as to the base-plate 8. The length section 1C of the leg to be measured extends from the upper side 80 of the base-plate 8 up to the circumferential plane U. Using the measuring tape loop 5, the circumference cC of the leg is determined at the measuring point cC, while simultaneously, using an ultrasonic distance measurement explained in more detail below, the length section 1C of the leg B is determined, which corresponds to the distance between the circumferential plane U and the upper side 80 of the base-plate 8.

FIG. 4 shows the schematic diagram of the ultrasonic distance measurement executed in the measurement of the calf according to FIG. 3. The measuring process for determining the distance between the circumferential plane U and the upper side 80 of the base-plate 80, which corresponds to the length section 1C between heel and calf, starts with emitting an ultrasonic signal 9 by the sender 6. The ultrasonic signal 9 propagates away from the sender 6 in a cone shape towards the base-plate 8 arranged in parallel to and at a distance from the lower side 10 of the housing 1, whereat the scanning beam 14 defined by the geometrical axis of the emission cone is oriented vertically to the front side 10. The ultrasonic signal 9 strikes the upper side 80 of the base-plate 8 facing the front-side 10, which upper side serves as the reflection surface. Part of the ultrasonic signal 9 striking the upper side 80 is reflected by the upper side 80 and is reflected back as an acoustic echo 15 towards the receiver 7, which receives and registers a portion of the reflected sound signal 15. Using an evaluation unit (not shown), the length section 1C can be determined from the time delay of the ultrasonic signal 9 in a known manner considering the double distance the measuring process is based on.

1. A device for determining measurements of human body parts (B), in particular the leg and hip measurements to be determined for a supply with compression stockings, at which first measuring means (5) are provided for the measurement of the circumference of the body part (B),
characterized in that
said device comprises second measuring means (6, 7), with which the length section (1C) of said body part (B) can be determined from a reference plane up to the circumferential plane (U).

2. The device according to claim 1,
characterized in that
with said second measuring means (6, 7) the distance measured in the direction of the longitudinal extension of said body part (B), generally measured vertically to said circumferential plane (U), can be measured between said circumferential plane (U) and said reference plane.

3. The device according to claim 1 or 2,
characterized in that
with said second measuring means (6, 7) the distance can be measured in a contactless manner.

4. The device according to claim 3,
characterized in that
said device comprises a sender (6) for emitting a physical measurement signal (9), said reference plane is designed as a reflection surface generally arranged in parallel to said circumferential plane (U), at which reflection surface said measurement signal (9) can be reflected, said device comprises a receiver (7) for receiving said measurement signal (9) reflected at said reflection surface, and an evaluation unit for determining the distance on the basis of the time delay of said measurement signal (9).

5. The device according to claim 4,
characterized in that
said measurement signal is an acoustic signal, in particular an ultrasonic signal (9).

6. The device according to claim 5,
characterized in that
said device comprises means to compensate for temperature-related variations in the sound velocity.

7. The device according to claim 4,
characterized in that
said measurement signal is an optical signal, in particular a laser signal.

8. The device according to any of claims 1 to 7,
characterized in that
said device comprises a display for displaying the measured values.

9. The device according to any of claims 1 to 8,
characterized in that
said device comprises an interface for data transfer, via which the measurement data comprising said circumferences and said respectively associated length sections can be transferred to a computer for further processing.

10. The device according to claim 9,
characterized in that
said interface is designed bidirectional, and via said interface data, in particular personal data of the persons to be measured, can be transferred from said computer to a signal processing unit in said device.

11. The device according to any of claims 1 to 10,
characterized in that
to said first measuring means comprise a measuring tape loop (5).

12. The device according to claim 11,
characterized in that
the circumference of said measuring tape loop (5) can be varied.

13. The device according to claim 12,
characterized in that
said measuring tape loop (5) is connected with a wind-up unit, which comprises a spring, which is tensioned upon pulling out said measuring tape loop (5).

14. The device according to claim 12,
characterized in that
said circumference of said measuring tape loop (5) can be varied using an electromotive drive.

15. The device according to any of claims 12 to 14,
characterized in that
means are provided to stop the pull-in movement of said measuring tape loop (5) upon reaching a predetermined contact pressure.

16. The device according to any of claims 11 to 15,
characterized in that
at said measuring tape loop (5), a fastening means (50) is provided, by which two loop sections (51, 52) can be releasably connected.

17. The device according to any of claims 11 to 16,
characterized in that
said measuring tape loop (5) is provided with an incremental gauge, and a scanning unit is provided, with which the circumference of said measuring tape loop can be measured by scanning the gauge.

18. The device according to claim 17,
characterized in that
said gauge is designed as punching or toothing.

19. The device according to claim 17 or 18,
characterized in that
said scanning unit is an optical scanning unit, which at least includes one light emitter and one photo-detector.

20. The device according to any of claims 17 to 19,
characterized in that
computing means are provided, with which the counting pulses generated by said scanning unit upon a pull-in movement of said measuring tape loop (5) and the counting pulses generated by said scanning unit upon an extension movement of said measuring tape loop (5) can be added up, and with which, based on the result of said addition, the current circumference of said measuring tape loop (5) can be determined.

21. The device according to claim 20,
characterized in that
said computing means comprise a differential encoder in connection with said photo-detector.

22. The device according to any of claims 11 to 21,
characterized in that
calibration means for zero-value calibration of said measuring tape loop (5) are provided.

23. The device according to any of claims 1 to 22,
characterized in that
said device comprises a housing (1).

24. The device according to claim 23,
characterized in that
at said housing (1), at least one inclination sensor for determining the relative position between said housing (1) and said reference plane is provided.

25. The device according to claim 23 or 24,
characterized in that
the side of said housing (1) facing said body part (B) has an inwardly concave outer contour complementary to said body part (B).

26. The device according to any of claims 1 to 25,
characterized in that
a particularly software-based instruction unit is provided, with which instructions for the execution of the measuring process can be transmitted to a user.

27. The device according to claim 26,
characterized in that
said instruction unit includes an acoustic announcement.

28. The device according to any of claims 1 to 27,
characterized in that
a foot length measuring unit is provided.

29. The device according to claim 28,
characterized in that
said housing (1) is designed as said foot length measuring unit.

30. The device according to claim 29,
characterized in that
said foot length measuring unit additionally comprises an extension part, which can be slid out of or folded out from said housing (1).

31. The device according to any of claims 1 to 30,
characterized in that
said device has an input unit, via which a signal processing unit and/or a data storage unit in said device can be supplied with information.

32. The device according to claim 31,
characterized in that
said input unit comprises a keyboard.

33. The device according to claim 31 or 32,
characterized in that
said device is designed to be able to execute the operations required for the processing of measurement signals and/or of data entered via said input unit as well as for the output and/or display of the measuring results autarkically and independently from external computers or peripheral devices.
